(19) United States
(12) Patent Application Publication
(10) Pub. No.: US 2005/0277868 A1
(43) Pub. Date: Dec. 15, 2005

(54) Electroporation Device and Method for Delivery to Ocular Tissue

(75) Inventors: Richard Heller, Tampa, FL (US); Mark J. Jaroszeski, Tampa, FL (US); Richard A. Gilbert, Tampa, FL (US); William H. Hauswirth, Gainesville, FL (US)

Correspondence Address: SMITH & HOPEN PA, 15950 BAY VISTA DRIVE, SUITE 220, CLEARWATER, FL, 33760, US

(73) Assignee: UNIVERSITY OF SOUTH FLORIDA, Tampa, FL (US)

(21) Appl. No.: 11/160,182

(22) Filed: Jun. 13, 2005

Related U.S. Application Data
(60) Provisional application No. 60/521,652, filed on Jun. 11, 2004.

(51) Int. Cl. A61N 1/30

(52) U.S. Cl. 604/21

(57) ABSTRACT

In accordance with the present invention is provided a device and method for delivering DNA for the purpose of gene therapy to specific regions within and around the eye.

### CROSS REFERENCE TO RELATED APPLICATIONS

This application claims priority to currently pending U.S. Provisional Patent Application No. 60/521,652, filed Jun. 11, 2004.

### BACKGROUND OF INVENTION

The effect of electromagnetic fields on cell membranes has been studied since the 1960's. Early research focused on describing observations that an applied electric field can break down cell membranes in vitro. Throughout the 1970's the topic was more common in the literature and continued to focus on describing the phenomenon that resulted from brief exposure to intense electric fields as well as the entry of exogenous molecules to the cell interior as a result of membrane breakdown. Applications began to emerge along with a better understanding of the reversible nature of cell membrane breakdown in the 1980's.

Prior research led to the current understanding that exposure of cells to intense electric fields for brief periods of time temporarily destabilized membranes. This effect has been described as a dielectric breakdown due to an induced transmembrane potential, and was termed “electroporation”, or “electropermeabilization”, because it was observed that molecules that do not normally pass through the membrane gain intracellular access after the cells were treated with electric fields. The porated state was noted to be temporary. Typically, cells remain in a destabilized state on the order of minutes after electrical treatment ceases.

The physical nature of electroporation makes it universally applicable. A variety of procedures utilize this type of treatment, which gives temporary access to the cytosol. These include production of monoclonal proteins, and genetic transformation. In addition, dyes and fluorescent molecules have been used to investigate the phenomenon of electroporation. A notable example of loading molecules into cells in vivo is electrochemotherapy. The procedure utilizes a drug combined with electric pulses as a means for loading tumor cells with an anticancer drug, and has been performed in a number of animal models and in clinical trials.

Protocols for the use of electroporation to load cells in vitro typically use a suspension of single cells or cells that are attached in a planar manner to a growth surface. In vivo electroporation is more complex because tissues are involved. Tissues are composed of individual cells that collectively make up a three-dimensional structure. In either case, the effects on the cell are the same. FIG. 1 illustrates details of the electroporation procedure commonly known in the art. Electrodes and electrode arrays for delivering electrical waveforms for therapeutic benefit, including inducing electroporation, have been described in the literature.

The loading of molecules by electroporation in vitro as well as in vivo is typically carried out by first exposing the cells or tissue of interest to a drug, as shown with reference to FIG. 2. The cells or tissue are then exposed to electric fields by administering one or more direct current pulses. Electrical treatment is conducted in a manner that results in a temporary membrane destabilization with minimal cytotoxicity. The intensity of electrical treatment is described by the magnitude of the applied electric field. This field is defined as the voltage applied to the electrodes divided by the distance between the electrodes. Electric field strengths ranging from 1000 to 5000 V/cm have been used and are specific to the cells or tissue under investigation. Pulses are usually rectangular in shape; however, exponentially decaying pulses have also been used. The duration of each pulse is called the pulse width. Molecule loading has been performed with pulse widths ranging from microseconds to milliseconds. The number of pulses delivered has ranged from one to eight. Typically, multiple pulses are utilized during electrical treatment.

For molecules to be delivered to the cell interior by electroporation, it is important that the molecule of interest be near the exterior of the cell membrane when a cell is in an electroporated state. It is also important to have molecules near substantially all cells within a treated tissue volume in order to provide efficient delivery to substantially all cells within the treatment volume.

Currently in the art, molecules are injected systemically or directly into the treatment site. No attempt is made to produce a specific distribution. These methods do not ensure that the distribution of molecules is sufficient to provide effective delivery to substantially all the cells.

Electropermeabilization of tumor cell membranes has been reported in the literature using applied electric pulses from surface electrodes in contact with the skin. Proteins and DNA can be transferred into the cells by incorporating either the protein or a DNA carrying a reporter gene. The efficiencies of transfer for the protein and plasmid were, respectively, 20 and 4%.

A first type of electrode known in the art for electroporation comprises two parallel-plate electrodes placed on opposite sides of a tumor of tissue of interest. Other electrodes known in the art at the present time comprise needles that are inserted into or around the tissue of interest. A third type comprises a planar arrangement of parallel wires that can be placed on the surface of the tissue.

Electrodes and methods known in the art do not provide molecular movement during the pre-electroporation time for electromigration, distribution, and post-electroporation time period when the cells are in a state of increased membrane permeability. The movement of molecules within the tissue is believed to affect an increase in the delivered quantity of molecules by enhancing movement into the cells.

Non-viral gene delivery has had some success in the art, but expression levels are low, have high variability and are not applicable to many tissue types. It has been shown that the addition of electroporation enhances the expression level of injected plasmid DNA.

Delivery of therapeutic genes to the ocular region, including the retina, vitreous, etc., has been limited to the use of viral delivery or direct injection. Adeno-Associated viruses have been used successfully to deliver to this area; however onset of expression is delayed. The use of electrically mediated gene delivery may allow for earlier expression and could enhance the treatment of several diseases of the ocular region.

Prior art electroporation studies have focused on the delivery of DNA to the cornea and have therefore utilized an electroporation device adapted to be effective with corneal tissue, similar to that described in U.S. Pat. No. 5,211,660 to Grasso. The Grasso patent describes a device that is only effective in the delivery of molecules to the superficial areas of the eye.

Accordingly, what is needed in the art is a device effective in deeper delivery to the tissues of the eye, including, but not limited to the retina and the vitreous that allows for more complete manipulation of the select molecule within the desired region of the eye.

### SUMMARY OF INVENTION

In accordance with the present invention is provided a device and method for delivering DNA for the purpose of gene therapy to specific regions within and around the eye.

The basic invention is a uniquely configured device that is designed specifically to enhance the delivery of molecules to the area in and around the eye. The prototype includes two electrodes on a flexible support that can be placed behind the retina. A third electrode is part of a hollow support, which can also be used to inject the molecule to the desired area. The electrodes can be positioned around the eye, including behind the retina or within the vitreous. One support will also act as an injection port to deliver the molecule to the desired area. The electrodes are independently addressable and will be used to administer the electric pulses.

Accordingly, the present invention provides a device and method for manipulating molecules within ocular tissue utilizing a desired electromagnetic field distribution within the ocular target tissue. As such, the present device and method affects in vivo gene delivery via electroporation and electromigration.

### BRIEF DESCRIPTION OF THE DRAWINGS

For a fuller understanding of the invention, reference should be made to the following detailed description, taken in connection with the accompanying drawings, in which:

FIG. 1 (Prior Art) Two-dimensional depiction of electroporation of a cell subjected to an electromagnetic field. Regions of membrane breakdown, depicted as pores, are formed at the ends of the cells facing the electrodes. Electromagnetic field exposure is achieved by applying a potential between electrodes − and +.

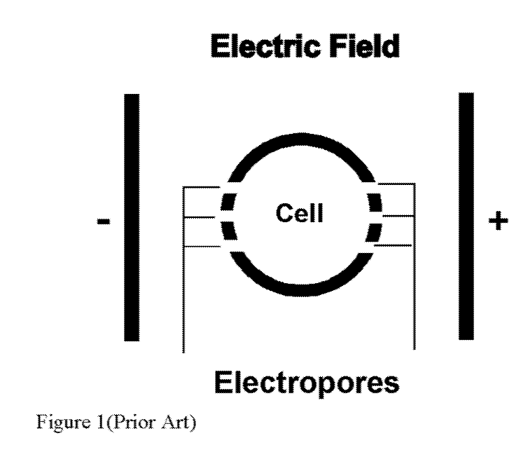

FIG. 2A-C (Prior Art) The process of delivering molecules by electroporation. FIG. 2A. A tumor cell in vitro or in vivo is exposed to the molecule of interest. FIG. 2B. Direct current pulses are administered to the cells to cause a temporary membrane destabilization that allows the molecules to more freely enter the cell interior. FIG. 2C. Cells return to their normal state after pulsation, leaving the drug within the cells.

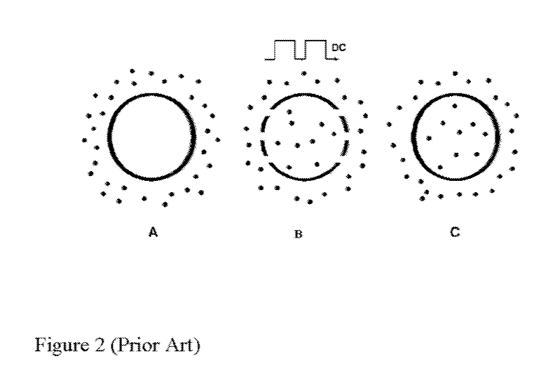

FIG. 3 is a diagrammatic illustration of the electrode assembly in accordance with the present invention.

### DETAILED DESCRIPTION OF THE PREFERRED EMBODIMENT

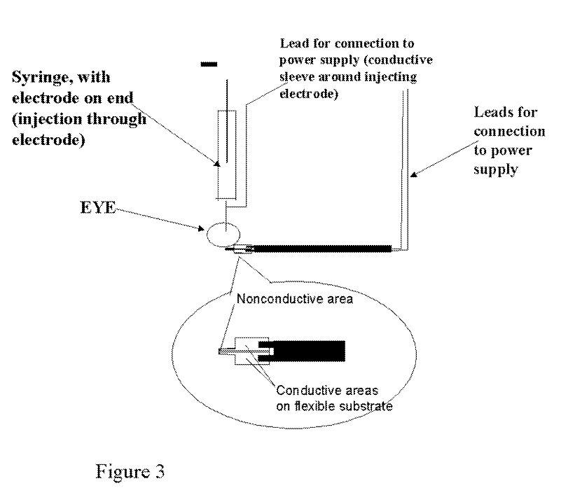

In accordance with the present invention, an electrode device for manipulating a molecule in vivo relative to an ocular target tissue is provided. The device with reference to FIG. 3 comprises a flexible support 10 and at least two discrete electrodes 15 extending away from and affixed to or incorporated into a support. An additional electrode 20 is contained on a separate support. This support can be hollow and thus could be used for injection of the molecule to be delivered to the target tissue. Each electrode is connectable in circuit communication with a respective portion of a source of electrical energy 25.

The electrodes are movable between a first position wherein they are a first distance apart and a second position wherein they are a second distance apart. The first distance is greater than the second distance, and the electrodes are biased to the first distance. This movability is for positioning the electrodes in a desired relation to a selected portion of the target tissue.

Means are also provided for maintaining a desired distance between the electrodes. Specifically, the means are adapted to restrain the electrodes from extending to the first position.

The electrodes are adapted to deliver, for example, alternating current, direct current, pulsed alternating current, pulsed direct current, high- and low-voltage alternating current with variable frequency and amplitude, variable direct current waveforms, variable alternating current signals biased with variable direct current waveforms, and variable alternating current signals biased with constant direct current.

In addition, these objects and others are attained by the present invention, a device for manipulating a molecule in vivo relative to a target tissue. The device comprises a support and at least one member affixed to and extending away from the support. The member has at least two discrete electrodes, each electrode in circuit communication with a respective portion of a source of electrical energy and therefore being differentially activatable.

The discrete electrodes are configured to establish a first electromagnetic field in vivo between selected electrodes sufficient to manipulate a molecule relative to a target tissue. The electrodes are further configured to establish a second, typically higher, electromagnetic field sufficient to cause transient permeability of a cell membrane within the target tissue.

Several embodiments of the methods of the present invention include the use of a device as described above to enhance the delivery of a molecule such as a bioactive molecule, nucleic acid, amino acid, polypeptide, protein, antibody, glyoprotein, enzyme, oligonucleotide, plasmid DNA, chromosome, or drug, although this list is not intended to be exhaustive or limiting. In a related embodiment, the device may be used to cause the electromigration of a least two components of a multicomponent reactive system into opposition to permit a reaction to occur at a desired target tissue site. The target tissue may comprise a tumor, an organ, or a wound site.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

1. An electroporation device effective for use with in vivo ocular tissue, the device comprising
a flexible support adapted for placement behind the retina; and
at least two independently addressable electrodes positioned on the flexible support.

2. The electroporation device of claim 1, further comprising an injection electrode to delivery a desired molecule to the target tissue.

3. The electroporation device of claim 1, further comprising a power supply in circuit communication with the at least two independently addressable electrodes.
